(19) United States
(12) Patent Application Publication
(10) Pub. No.: US 2015/0182620 A1
(43) Pub. Date: Jul. 2, 2015

(54) Polypeptides for use in the Prophylactic Treatment of Allergic Asthma

(71) Applicants: INSERM (Institut de la Sante et de la Recherche Medicale), Paris (FR); Universite de Nantes, Nantes (FR); Centre Hospitalier Universitaire de Nantes, Nantes Cedex (FR); Centre National de la Recherche Scientifique (CNRS), Paris (FR); Medizinische Universitaet Wien, Wien (AT)

(72) Inventors: Antoine Magnan, Nantes Cedex 1 (FR); David Lair, Nantes Cedex 1 (FR); Susanne Vrtala, Vienna (AT)

(21) Appl. No.: 14/408,003

(22) PCT Filed: Jun. 17, 2013
(86) PCT No.: PCT/EP2013/062511

§ 371 Date: Dec. 15, 2014

(30) Foreign Application Priority Data
Jun. 15, 2012 (EP) 12305680.6

(51) Int. Cl. A61K 39/35; C07K 14/435

(52) U.S. Cl. CPC A61K 39/35; C07K 14/43531; A61K 2039/53

(57) ABSTRACT

The present invention relates to a polypeptide derived from a mite allergen (Der p 2) useful for the prophylactic treatment of allergic asthma.

### FIELD OF THE INVENTION

The invention relates to a polypeptide derived from a mite allergen (Der p 2) useful for the prophylactic treatment of allergic asthma.

### BACKGROUND OF THE INVENTION

Allergic asthma is a chronic respiratory disease affecting 300 million people worldwide (Global Initiative for Asthma (GINA), 2011). The number of individuals with asthma has doubled during the last ten years and around 250,000 people die prematurely each year due to this condition. In the majority of cases, asthma is caused by an abnormal reactivity against some environmental antigens, also called allergens. In Western Europe, the prevalence of atopic diseases (including asthma and rhinitis) is more than 30%, thus allergic diseases are considered to be an important public health issue. Considering the pathophysiological aspects, allergic asthma is a bronchial inflammatory disease resulting from the exposition of a predisposed subject to different allergens. In Europe and the USA, the most-commonly encountered species of mite is Dermatophagoides pteronyssimus (Der p). Asthma patients are usually treated with corticosteroids, which, however, only suspend the disease and are associated with deleterious side effects. An alternative treatment for allergic asthma is based on a specific immunotherapy protocol: the repeated administration of increasing doses of allergen to induce hyposensitivity, and hence reduced symptoms when another subsequent exposure to this allergen occurs. Nevertheless, the efficacy of immunotherapy remains limited, and its efficacy is very variable between patients.

It is therefore necessary to have a prophylactic vaccine to prevent allergic asthma and more particularly asthma exacerbations caused by allergens.

The major allergen of the house-dust mite Dermatophagoides pteronyssimus Der p 2 is a 146-amino acid protein which is further processed into a secreted mature form of 129 amino acids) after cleavage of the signal peptide (amino acids 1-17). Until now, Der p 2 has only been proposed for desensitizing a patient allergic to a house dust mite patient (e.g. a patient allergic to Dermatophagoides pteronyssinus and/or Dermatophagoides farina) but has never been shown or suggested as useful for prophylactic treatment of allergic asthma in allergic or atopic patients who are not yet asthmatic patients.

Recently, Chen et al. 2008 described that two recombinant fragments of Der p 2 (rDer p 2.1 consisting of amino acids 1 to 53 of the mature form Der p 2 and rDer p 2.2 consisting of amino acids 54 to 129) exhibited less in vivo allergenic activity and allergenicity than the Derp 2 allergen but preserved immunogenicity and may represent candidates for specific immunotherapy of house-dust mite allergy.

However, the effects of the Derp 2 allergen or derivatives thereof such as rDer p 2.1 and rDer p 2.2 on the respiratory function have never been studied. Indeed, only the induction of IgE responses has been studied after immunization. Moreover, the action of the derivatives rDer p 2.1 and rDer p 2.2 has never been studied in vivo in an allergic asthma mouse model.

### SUMMARY OF THE INVENTION

The present invention relates to an isolated polypeptide comprising a biologically active fragment of the allergen of the house-dust mite Dermatophagoides pteronyssinus Der p 2, wherein said fragment comprises:

a) the sequence consisting of amino acids 18 to 70 of SEQ ID NO: 1;

b) a sequence at least 80% identical to the sequence of (a);

c) at least six consecutive amino acids of the sequence of (a) or (b);

for use in the prophylactic treatment of allergic asthma in a patient in need thereof.

### DETAILED DESCRIPTION OF THE INVENTION

Now, the inventors have surprisingly found that only the administration of rDer p 2.1 is useful as a prophylactic treatment of allergic asthma, and particularly for preventing asthma exacerbations. In particular, only rDer p 2.1 improves the respiratory function, and decreases the level of inflammatory cells (whereas rDer p 2.2 does not provide a prophylactic benefit since this fragment does not decrease pulmonary inflammation). Unexpectedly, they have also shown that immunization with native Der p 2 induce a degradation of respiratory function compare to control mice and an increase of inflammation cells influx in BALF leading to preclude its utilization in prophylactic treatment of allergic asthma. Moreover, they have shown a bystander effect obtained by immunization with rDerp 2.1 since this peptide improves the respiratory function in Der f allergic mice.

### Polypeptides Derived from Der p 2 Allergen and Uses Thereof

The polypeptides according to the invention, for use in the prophylactic treatment of allergic asthma as further described herein, are polypeptides comprising or consisting of a biologically active fragment of the house-dust mite Dermatophagoides pteronyssinus Der p 2.

As used herein, the term “Der p 2” refers to the major allergen of the house-dust mite (HDM) Dermatophagoides pteronyssimus which is recognized by approximatively 90% of mite allergic patients. The naturally occurring Der p 2 consists of the amino acid sequence consisting of amino acids 18 to 146 of SEQ ID NO: 1. Thus, SEQ ID NO: 1 corresponds to the sequence of Der p 2+the signal peptide that is referenced with the UniProtKB/Swiss-Prot Accession number P49278.

As used herein, the term “Der p 2.1” refers to the polypeptide consisting of the amino acid sequence 18 to 70 of SEQ ID NO: 1. As used, the term “Der p 2.2” refers to the polypeptide consisting of the amino acid sequence 71 to 146 of SEQ ID NO: 1.

A first aspect of the invention relates to an isolated polypeptide comprising a biologically active fragment of the allergen of the house-dust mite Dermatophagoides pteronyssinus Der p 2, wherein said fragment comprises:

a) the sequence consisting of amino acids 18 to 70 of SEQ ID NO: 1;

b) a sequence at least 80% identical to the sequence of (a);

c) at least six consecutive amino acids of the sequence of (a) or (b);

for use in the prophylactic treatment of allergic asthma in a patient in need thereof.

The sequence of the polypeptide of the invention is represented in Table A below:

TABLE A
Amino acid sequence of the polypeptide of the invention.

| SEQ ID | | Nomenclature used in |
|---|---|---|
| number | Sequence | the patent application |
| Polypeptide | MMYKILCLSLLVAAVARDQVDVKDCA | Mite group 2 allergen |
| SEQ ID NO: 1 | NHEIKKVLVPGCHGSEPCIIHRGKPFQLE | Der p 2 (Complete |
| | AVFEANQNTKTAKIEIKASIDGLEVDVP | form before processing |
| | GIDPNACHYMKCPLVKGQQYDIKYTWN | into a mature form by |
| | VPKIAPKSENVVVTVKVMGDDGVLACA | cleavage of the signal |
| | IATHAKIRD | peptide) |

By a polypeptide having an amino acid sequence at least, for example, 95% “identical” to a query amino acid sequence of the present invention, it is intended that the amino acid sequence of the subject polypeptide is identical to the query sequence except that the subject polypeptide sequence may include up to five amino acid alterations per each 100 amino acids of the query amino acid sequence. In other words, to obtain a polypeptide having an amino acid sequence at least 95% identical to a query amino acid sequence, up to 5% (5 of 100) of the amino acid residues in the subject sequence may be inserted, deleted, or substituted with another amino acid.

In the frame of the present application, the percentage of identity is calculated using a global alignment (i.e., the two sequences are compared over their entire length). Methods for comparing the identity and homology of two or more sequences are well known in the art. The <<needle>> program, which uses the Needleman-Wunsch global alignment algorithm (Needleman and Wunsch, 1970 J. Mol. Biol. 48:443-453) to find the optimum alignment (including gaps) of two sequences when considering their entire length, may for example be used. The needle program is for example available on the ebi.ac.uk world wide web site. The percentage of identity in accordance with the invention is preferably calculated using the EMBOSS::needle (global) program with a “Gap Open” parameter equal to 10.0, a “Gap Extend” parameter equal to 0.5, and a Blosum62 matrix.

Polypeptides consisting of an amino acid sequence “at least 80%, 85%, 90%, 95%, 96%, 97%, 98% or 99% identical” to a reference sequence may comprise mutations such as deletions, insertions and/or substitutions compared to the reference sequence. The polypeptide consisting of an amino acid sequence at least 80%, 85%, 90%, 95%, 96%, 97%, 98% or 99% identical to a reference sequence may correspond to an allelic variant of the reference sequence. It may for example only comprise substitutions compared to the reference sequence. The substitutions preferably correspond to conservative substitutions as indicated in the table below.

| Conservative substitutions | Type of Amino Acid |
|---|---|
| Ala, Val, Leu, Ile, Met, Pro, Phe, Trp | Amino acids with aliphatic hydrophobic side chains |
| Ser, Tyr, Asn, Gln, Cys | Amino acids with uncharged but polar side chains |
| Asp, Glu | Amino acids with acidic side chains |
| Lys, Arg, His | Amino acids with basic side chains |
| Gly | Neutral side chain |

In one particular embodiment, the isolated polypeptide for use in the prophylactic treatment of allergic asthma in a patient in need thereof, comprises a biologically active fragment of at most 100 consecutive amino acids of the allergen of the house-dust mite Dermatophagoides pteronyssinus Der p 2, wherein said fragment comprises:

a) the sequence consisting of amino acids 18 to 70 of SEQ ID NO: 1;

b) a sequence at least 80% identical to the sequence of (a);

c) at least six consecutive amino acids of the sequence of (a) or (b).

The polypeptides according to the invention, for use in the prophylactic treatment of allergic asthma as further described herein, encompass polypeptides comprising or consisting of fragments of Der p 2 allergen, provided the fragments are biologically active.

In the frame of the invention, the biologically active fragment may for example comprise at least 6, 8, 10, 12, 15, 18, 20, 25, 30, 35, 40, 45, 50, 55, 60, 65, 70, 75, 80, 85, 90, 95 or 100 consecutive amino acids of Der p 2. According to the invention, a polypeptide comprising a biologically active fragment of at most 100 consecutive amino acids of Der p 2 does not contain more than 100 consecutive amino acids of Der p 2. Indeed, “a polypeptide comprising a biologically active fragment of at most 100 consecutive amino acids of Der p 2” according to the invention refers to a polypeptide of at most 100 consecutive amino acids of Der p 2. Accordingly, said “polypeptide comprising or consisting of a biologically active fragment of Der p 2” is not the full length mature Der p 2 protein.

By “biological activity” of a fragment of Der p 2 is meant (i) the capacity to reduce airway hyperresponsiveness (in particular Der p-induced airway hyperresponsiveness); and/or (ii) the capacity to reduce pulmonary inflammation; and/or (iii) the capacity to decrease the number of inflammatory cells (in particular Th2-cytokine-producing cells); and/or (iv) the capacity to reduce inflammatory cytokine levels; and/or (v) the capacity of improving respiratory function.

The skilled in the art can easily determine whether a polypeptide fragment of Der p 2 is biologically active. To check whether the newly generated polypeptides decrease the airway hyperresponsiveness in the same way than the initially characterized polypeptide rDer p 2.1 a lung function measurement and broncho-alveolar lavage (BAL) cytology in a mouse model of Der p-induced allergy mimicking asthma and allergen-driven exacerbation (such as described in Example) may be performed with each polypeptide. Additionally, a time-course and a dose-response performed in said mouse model of Der p-induced allergy mimicking asthma and allergen-driven exacerbation will determine the optimal conditions for each polypeptide.

As used herein, a “biologically active” fragment refers to a fragment exhibiting at least one, preferably all, of the biological activities of Derp 2.1, provided the biologically active fragment retains the capacity of decreasing the airway hyperresponsiveness and/or preventing asthma exacerbations. The biologically active fragment may for example be characterized in that it is capable of improving respiratory function when assessed by plethysmography (see Example and FIG. 3) and/or decreasing pulmonary inflammation (e.g. decrease of the number of inflammatory cells in lung or in BALF (broncho-alveolar lavage fluid) (when assessed through lund cytology or BALF cytology) (see Example and FIGS. 4 and 5).

The inventors have identified that only the fragment of Derp 2.1 (unlike to Der p 2.2) both known as exhibiting less in vivo allergenic activity and allergenicity than the full-length Derp 2 allergen but preserving immunogenicity) enables to decrease airway hyperresponsiveness and prevent asthma exacerbations (by decreasing the pulmonary inflammation via a decrease of the number of inflammatory cells, in particular Th2-cytokine-producing cells involved in allergic asthma (e.g. eosinophils) and a decrease of the level of inflammatory cytokines (e.g.).

Therefore, the present invention provides an isolated polypeptide comprising or consisting of a biologically active fragment of Der p 2, wherein said fragment comprises or consists of:

a) the sequence consisting of amino acids 18 to 70 of SEQ ID NO: 1;

b) a sequence at least 80, 85, 90, 95, 96, 97, 98 or 99% identical to the sequence of (a);

c) at least 6, 8, 10, 12, 15, 18, 20, 25, 30, 35, 40, 45, 50, 55, 60, 65, 70, 75, 80 or 85, 90, 95 or 100 consecutive amino acids of the sequence of (a) or (b);

for use in the prophylactic treatment of allergic asthma in a patient in need thereof.

In a particular embodiment, the polypeptide for use consists of
a) the sequence consisting of amino acids 18 to 70 of SEQ ID NO: 1;
b) a sequence at least 80, 85, 90, 95, 96, 97, 98 or 99% identical to the sequence of (a);
c) at least 6, 8, 10, 12, 15, 18, 20, 25, 30, 35, 40, 45, 50 consecutive amino acids of the sequence of (a) or (b).

By an “isolated” polypeptide, it is intended that the polypeptide is not present within a living organism, e.g. within a house-dust mite such as Dermatophagoides pteronyssimus.

The isolated polypeptide is preferably purified. The term “purified” employed herein means that the polypeptide of the invention (e.g. Der p 2.1) contains less than 5% other components from the host namely (poly)peptides contaminants.

The polypeptides of the invention may be produced by any method well known in the art (e.g. chemical synthesis or recombinant techniques).

Examples of chemical synthesis technologies are solid phase synthesis and liquid phase synthesis. As a solid phase synthesis, for example, the amino acid corresponding to the C-terminus of the polypeptide to be synthesized is bound to a support which is insoluble in organic solvents, and by alternate repetition of reactions, one wherein amino acids with their amino groups and side chain functional groups protected with appropriate protective groups are condensed one by one in order from the C-terminus to the N-terminus, and one where the amino acids bound to the resin or the protective group of the amino groups of the polypeptides are released, the polypeptide chain is thus extended in this manner. Solid phase synthesis methods are largely classified by the tBoc method and the Fmoc method, depending on the type of protective group used. Typically used protective groups include tBoc (t-butoxycarbonyl), Cl—Z (2-chlorobenzyloxycarbonyl), Br—Z (2-bromobenzyloyycarbonyl), Bzl (benzyl), Fmoc (9-fluorenylmethoxycarbonyl), Mbh (4,4′-dimethoxydibenzhydryl), Mtr (4-methoxy-2,3,6-trimethylbenzenesulphonyl), Trt (trityl), Tos (tosyl), Z (benzyloxycarbonyl) and Clz-Bzl (2,6-dichlorobenzyl) for the amino groups; NO2 (nitro) and Pmc (2,2,5,7,8-pentamethylchromane-6-sulphonyl) for the guanidino groups); and tBu (t-butyl) for the hydroxyl groups). After synthesis of the desired peptide, it is subjected to the de-protection reaction and cut out from the solid support. Such peptide cutting reaction may be carried with hydrogen fluoride or tri-fluoromethane sulfonic acid for the Boc method, and with TFA for the Fmoc method.

Alternatively, the polypeptide may be synthesized using recombinant techniques. In this case, a nucleic acid encoding a polypeptide according to the invention (further referred to as “a nucleic acid according to the invention”) is cloned into an expression vector. The nucleic acid of the invention is preferably placed under the control of expression signals (e.g. a promoter, a terminator and/or an enhancer) allowing its expression. The expression vector is then transfected into a host cell (e.g. a human, CHO, mouse, monkey, fungal or bacterial host cell), and the transfected host cell is cultivated under conditions suitable for the expression of the polypeptide. For instance, in the section “Examples”, the term “rDer p 2.1” designates the recombinant Der p 2.1 polypeptide expressed by Escherichia coli (E. coli). The term “expression vector” is meant to include any type of genetic construct containing a nucleic acid coding for a gene product in which part or all of the nucleic acid encoding sequence is capable of being transcribed.

The method of producing the polypeptides of the invention may optionally comprise the steps of purifying said polypeptide, chemically modifying said polypeptide, and/or formulating said polypeptide into a pharmaceutical composition (e.g. a vaccine).

In one embodiment, the polypeptides of the invention may comprise a tag. A tag is an epitope-containing sequence which can be useful for the purification of the polypeptides. It is attached to by a variety of techniques such as affinity chromatography, for the localization of said peptide or polypeptide within a cell or a tissue sample using immunolabeling techniques, the detection of said peptide or polypeptide by immunoblotting etc. Examples of tags commonly employed in the art are the GST (glutathion-S-transferase)-tag, the FLAG™-tag, the Strep-tag™, V5 tag, myc tag, His tag (which typically consists of six histidine residues), etc.

In another embodiment, the polypeptides of the invention may comprise chemical modifications improving their stability and/or their biodisponibility. Such chemical modifications aim at obtaining polypeptides with increased protection of the polypeptides against enzymatic degradation in vivo, and/or increased capacity to cross membrane barriers, thus increasing its half-life and maintaining or improving its biological activity. Any chemical modification known in the art can be employed according to the present invention. Such chemical modifications include but are not limited to:
replacement(s) of an amino acid with a modified and/or unusual amino acid, e.g. a replacement of an amino acid with an unusual amino acid like Nle, Nva or Orn; and/or
modifications to the N-terminal and/or C-terminal ends of the peptides such as e.g. N-terminal acylation (preferably acetylation) or desamination, or modification of the C-terminal carboxyl group into an amide or an alcohol group;
modifications at the amide bond between two amino acids: acylation (preferably acetylation) or alkylation (preferably methylation) at the nitrogen atom or the alpha carbon of the amide bond linking two amino acids;
modifications at the alpha carbon of the amide bond linking two amino acids such as e.g. acylation (preferably acetylation) or alkylation (preferably methylation) at the alpha carbon of the amide bond linking two amino acids.
chirality changes such as e.g. replacement of one or more naturally occurring amino acids (L enantiomer) with the corresponding D-enantiomers;
retro-inversions in which one or more naturally-occurring amino acids (L-enantiomer) are replaced with the corresponding D-enantiomers, together with an inversion of the amino acid chain (from the C-terminal end to the N-terminal end);
azapeptides, in which one or more alpha carbons are replaced with nitrogen atoms; and/or
betapeptides, in which the amino group of one or more amino acid is bonded to the β carbon rather than the α carbon.

Another strategy for improving biological activity is the utilization of water-soluble polymers. Various water-soluble polymers have been shown to modify biodistribution, improve the mode of cellular uptake, change the permeability through physiological barriers; and modify the rate of clearance from the body. To achieve either a targeting or sustained-release effect, water-soluble polymers have been synthesized that contain drug moieties as terminal groups, as part of the backbone, or as pendent groups on the polymer chain.

Polyethylene glycol (PEG) has been widely used as a drug carrier, given its high degree of biocompatibility and ease of modification. Attachment to various drugs, proteins, and liposomes has been shown to improve residence time and decrease toxicity. PEG can be coupled to active agents through the hydroxyl groups at the ends of the chain and via other chemical methods; however, PEG itself is limited to at most two active agents per molecule. In a different approach, copolymers of PEG and amino acids were explored as novel biomaterials which would retain the biocompatibility properties of PEG, but which would have the added advantage of numerous attachment points per molecule (providing greater drug loading), and which could be synthetically designed to suit a variety of applications.

Another aspect of the invention relates to a method for prophylactically treating allergic asthma comprising administering to a patient in need thereof an effective amount of a polypeptide according to the invention as described above.

The polypeptides are administered in an “effective amount”, i.e. in an amount sufficient to prophylactically treat the allergic asthma. It will be appreciated that this amount will vary with the effectiveness of therapeutic agent(s) employed, with the nature of any carrier used, with the seriousness of the disease and the age of the patient. The determination of appropriate amounts for any given composition is within the skill in the art, through standard series of tests designed to assess appropriate therapeutic levels.

By “patient in need thereof” is meant an allergic patient, in particular a mite-allergic patient or an atopic patient without asthmatic manifestation. In the frame of the present invention, the patient preferably is a human individual.

In one particular, the mite-allergic patient is allergic to one or more allergen selected from the group consisting of Dermatophagoides pteronyssinus 1 (Der p 1), Dermatophagoides pteronyssinus 2 (Der p 2), Dermatophagoides farinae 1 (Der f 1) and Dermatophagoides farinae 2 (Der f 2). According to the invention, the patient is an allergic patient without asthmatic manifestations.

Alternatively, the patient in need thereof may be allergic to any other environmental allergen. Allergens are well-known to the skilled in the art. Common environmental allergens which induce allergic diseases are found in pollen (e.g. tree, herb, weed and grass pollen allergens), food, animal danders, hair and/or saliva (from e.g. dog, cat, horse, rat, mouse etc.), molds, fungal spores and venoms (e.g. insect or batracian venom).

In another particular, the patient in need thereof is an atopic patient. Atopy is the genetic predisposition of an individual to produce high quantities of IgE in response to allergens in the environment (pollens, house dust mites, moulds, cat dander, foods etc). Atopy can have a hereditary component, although contact with the allergens should occur before the hypersensitivity reaction may develop. However, an atopic patient will not necessarily progress to an asthmatic state (not all atopic people start developing respiratory symptoms). According to the invention, the patient is an atopic patient without asthmatic manifestations. However, said patient may have a tendency to have food or cutaneous allergy, and other symptoms marked by their hypersensitivity state.

### A Nucleic Acid According to the Invention and Uses Thereof

In a second aspect, the invention also provides nucleic acids encoding and/or vectors expressing a polypeptide comprising or consisting of a biologically active fragment of Der p 2 as described above for use in the prophylactic treatment of allergic asthma in a patient in need thereof.

Such nucleic acids or vectors find use when a polypeptide comprising or consisting of a biologically active fragment of Der p 2 is intended to be administered to a patient in the frame of a gene therapy. In this case, the nucleic acid is preferably present on a expression vector, on which the sequence coding for the polypeptide is placed under the control of expression signals (e.g. a promoter, a terminator and/or an enhancer) allowing its expression. The vector may for example correspond to a viral vector such as an adenoviral or a lentiviral vector.

Alternatively, a synthetic vector may be used in combination with nucleic acids encoding a polypeptide comprising or consisting of a biologically active fragment of Der p 2.

Such synthetic vector may be specific chemical molecules may be tetrafunctional block copolymers which have been shown able to safely increase the transfection efficiency of reporter or therapeutic genes in lung. This class of vector has been proposed for intracellular delivery of nucleic acids (WO 03/066104).

Accordingly, a tetrafunctional copolymer suitable for the invention may be a compound of formula (I):

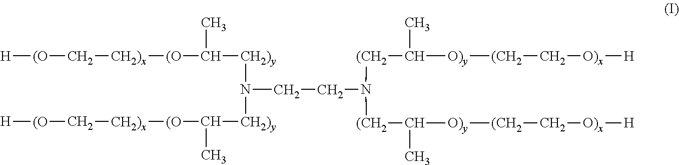

wherein x and y represent, independently of one another, an integer of between 1 and 500 with x having a value such that said molecule comprises at least 40% by weight of ethylene oxide units.

The invention may also be implemented with derivatives of compounds of formula (I).

For the purpose of the present invention, the term “derivative” is intended to cover compounds which have the chemical structure of general formula I but which also carry secondary chemical or biological functions or entities capable of conferring on them complementary properties. Particularly representative of these derivatives are compounds of general formula (I) in which there is also as least one intra- or extracellular targeting unit. By way of non-limiting illustration of these targeting units, mention may more particularly be made of peptides carrying a nuclear localization sequence, or peptides which recognize receptors present at the surface of certain cells.

A compound of general formula (I) has preferably no more than 85% by weight of ethylene oxide units.

A compound of general formula (I) has in particular approximately between 40 and 80% by weight of ethylene oxide units.

According to a preferred variant of the invention, the molecules of compounds of general formula (I) also have a molecular weight of at least 800 g/mol, and more preferably of between 1000 and 25 000 g/mol.

According to a preferred embodiment of the invention, the compounds of general formula have an EO/PO unit ratio of between 0.5 and 1.5, and preferably of the order of 1±0.2.

As compounds of general formula (I) that are most particularly suitable for the present invention, mention may more particularly be made of molecules having, respectively, a molecular weight of 1650 g for an EO/PO ratio of 15:16 (for example poloxamine 304), of 5500 g/mol for an EO/PO ratio of 50:56 (for example poloxamine 704) and of 6700 g/mol for an EO/PO ratio of 61:68 (for example poloxamine 904).

According to a preferred embodiment a compound of formula (I) may be selected in the group consisting of poloxamine 304, 704, 904, and mixture thereof.

According to a preferred embodiment, the composition is free of sodium phosphate and/or of glucose.

A tetrafunctional copolymer suitable for the invention is used in a cationic form.

A compound of general formula (I) is preferably used in the form of one of its salts, and more preferably in a cationic form. To do this, the composition claimed combines with said compound a preferably mineral salt, and more preferably an alkali metal salt or an alkaline-earth metal salt. It may in particular be chosen from sodium chloride, potassium chloride or lithium chloride and sodium thiocyanate, or more preferably calcium chloride (CaCl₂) or magnesium chloride (MgCl₂).

This salt may be introduced in isotonic, hypotonic or hypertonic amount.

The inventors have also established the advantage of controlling the pH and/or the ionic composition of the formulation, in order to be sure that the copolymer of formula (I) is in its cationic form.

A pH of 6.5 to 8, preferably 7 to 7.8, more preferably 7.4, proves to be particularly advantageous.

According to a preferred embodiment of the invention, the composition is formulated in a medium referred to as Tyrode's (medium containing 3 mM CaCl₂, 2 mM MgCl₂, 6 mM KCl, 140 mM NaCl, 10 mM glucose and 10 mM Hepes, pH 7.4) (Tyrode Pharmacology. Philadelphia, 1908, 2nd edition, 1912). The presence of the Tyrode's makes it possible in particular to control the ionic composition of the formulation and the pH and, consequently, the use of the compound of formula (I) in a cationic form.

The preparation of a nucleic acid molecule with a poloxamine in a salt medium, and in particular in presence of Tyrode medium may be made as described in WO 03/066104.

Another aspect of the invention relates to a method for prophylactically treating allergic asthma comprising administering to a patient in need thereof an effective amount of a nucleic acid encoding and/or a vector expressing a polypeptide according to the invention as described above.

### Vaccine and Uses Thereof

In a third aspect, the invention further relates to a vaccine comprising a polypeptide as described here above or a nucleic acid sequence as described here above for use in the prophylactic treatment of allergic asthma in a patient in need thereof.

The prophylactic administration of the vaccine of the invention should serve to prevent allergic asthma and in particular asthma exacerbations in a patient in need thereof. In a preferred embodiment, said patient, preferably human, is a mite-allergic patient.

When provided therapeutically, the immunising composition of the invention is provided to enhance the patient's own immune response to the mite allergen in particular house-dust mite Dermatophagoides pteronyssimus.

In one particular, the mite-allergic patient is allergic to one or more allergen selected from the group consisting of Dermatophagoides pteronyssinus 1 (Der p 1), Dermatophagoides pteronyssinus 2 (Der p 2), Dermatophagoides farinae 1 (Der f 1) and Dermatophagoides farinae 2 (Der f 2).

Alternatively, the patient in need thereof may be allergic to any other environmental allergen. Allergens are well-known to the skilled in the art. Common environmental allergens which induce allergic diseases have been above-described.

Further to polypeptide or nucleic acid sequence, a vaccine of the invention may comprise one or more pharmaceutically acceptable carriers and, optionally, other therapeutic ingredients. The carrier(s) must be “acceptable” in the sense of being compatible with the other ingredients of the vaccine and not deleterious to the recipient thereof.

Formulation of the vaccines of the invention can be accomplished using art recognized methods. The amount of vaccines of the invention to be administered to a patient and the regime of administration can be determined in accordance with standard techniques well known to those of ordinary skill in the pharmaceutical arts taking into consideration such factors as the adjuvant (if present), the age, sex, weight, and condition of the particular patient and the route of administration. The administration of the vaccine is usually in a single dose. Alternatively, the administration of the vaccine of the invention is made a first time (initial vaccination), followed by at least one recall (subsequent administration), with the vaccine.

The vaccines of the invention can be formulated in any suitable manner. In general, the vaccines of the present invention can be administered orally, nasally, nasopharyngeally, parenterally, enterically, gastrically, topically, transdermally, subcutaneously, intramuscularly, in tablet, solid, powdered, liquid, aerosol form, locally or systemically, with or without added carriers.

A vaccine of the invention can be administered as a capsule or tablet, as a dry powder or in liquid form. Administration can for example be achieved by injection (cg, subcutaneous, or intravenous), orally such as by dosage unit form (e.g., tablet, capsule or dosed liquid form), or by inhalation.

Vaccines suitable for intravenous, intradermal, intramuscular, subcutaneous, or intraperitoneal administration conveniently comprise sterile aqueous solutions of the active agent with solutions which are preferably isotonic with the blood of the recipient. Such vaccines may be conveniently prepared by dissolving solid active ingredient in water containing physiologically compatible substances such as sodium chloride (e.g. 0.1-2.0M), glycine, and the like, and having a buffered pH compatible with physiological conditions to produce an aqueous solution, and rendering said solution sterile. These may be present in unit or multi-dose containers, for example, sealed ampoules or vials.

When oral preparations are desired, the vaccines may be combined with typical carriers, such as lactose, sucrose, starch, talc magnesium stearate, crystalline cellulose, methyl cellulose, carboxymethyl cellulose, glycerin, sodium alginate or gum arabic among others.

The vaccines of the invention may incorporate a stabilizer. Illustrative stabilizers are polyethylene glycol, proteins, saccharides, amino acids, inorganic acids, and organic acids which may be used either on their own or as admixtures.

According to a particular embodiment, combinatorial methods may be implemented according to the present invention. They involve the administration of at least two agents to a patient, the first of which is a vaccine according to the invention, and the second of which is a second therapeutic agent.

The combinatorial therapy methods of the present invention can result in a greater than additive effect, providing therapeutic benefits where neither the vaccine nor second therapeutic agent administered in an amount that is alone effective for treatment of asthma.

In the methods, the vaccine and the second therapeutic agent can be administered concurrently or successively. As used herein, the vaccine and the second therapeutic agent are said to be administered concurrently if they are administered to the patient on the same day, for example, simultaneously, or 1, 2, 3, 4, 5, 6, 7 or 8 hours apart. In contrast, the vaccine and the second therapeutic agent are said to be administered successively if they are administered to the patient on the different days, for example, the vaccine and the second therapeutic agent can be administered at a 1-day, 2-day or 3-day, one-week, 2-week or monthly intervals. In the methods of the invention, administration of the vaccine can precede or follow administration of the second therapeutic agent.

As a non-limiting example, the vaccine and second therapeutic agent can be administered concurrently for a period of time, followed by a second period of time in which the administration of the vaccine and the second therapeutic agent is alternated.

Most people with persistent asthma use a combination of long-term control medications and quick-relief medications, taken with a hand-held inhaler. Asthma symptoms triggered by allergens are also treated with allergy medications.

Accordingly, suitable second therapeutic agents include long-term control medications, quick-relief medications, and allergy medications.

The second therapeutic agent may be corticosteroids, short-acting beta-2 agonists (SABAs) such as albuterol or albuterol sulfate, anti-histamines, for example.

The invention also relates to a method for immunization of a patient in need thereof, comprising administering a vaccine comprising a polypeptide or a nucleic acid sequence as described here above.

The invention will be further illustrated by the following figures and examples. However, these examples and figures should not be interpreted in any way as limiting the scope of the present invention.

### FIGURES

FIG. 1: Der p asthmatic model and immunization strategy.

FIG. 2: A. BALF's cells concentration 3 days (Day 37) after last challenge in Dermatophagoides pternyssinus allergic asthma model. B. Histologic grade 3 days (Day 37) after last challenge in Dermatophagoides pternyssinus allergic asthma model. C. Respiratory function 3 day (Day 37) after last challenge in Dermatophagoides pternyssinus allergic asthma model.

FIG. 3: A. Respiratory function 1 day (Day 35) or 3 days (Day 37) after the last challenge with Der p extract in mice immunized with rDer p 2.1 or Bet v-1. B. Respiratory function 1 day after the last challenge with Der p extract in mice immunized with rDer p 2.1, rDer p 2.2, Der p 2 native, rDer p 2.1+2.2 or Control (Bet v-1 or Bet v-2).

FIG. 4: Lung cytology 1 day after the last challenge with Der p extract in mice immunized with rDer p 2.1 or Bet v-1.

FIG. 5: A. BALF cytology 1 day after the last challenge with Der p extract in mice immunized with rDer p 2.1 or Bet v-1. B. BAL cell's concentration 3 days after the last challenge with Der p extract (asthmatic mice) or PBS (controls mice) in mice immunized with rDer p 2.1 or Bet v-1.

FIG. 6: BALF cytology 1 day after the last challenge with Der p extract in mice immunized with rDer p 2.1, rDer p 2.2, native Der p 2, combination of rDerp 2.1 and rDerp 2.2 or Bet v-1.

FIG. 7: Respiratory function 1 day after the last challenge with Der f extract in Der f allergic mice immunized twice with rDer p 2.1 or Bet v-1.

FIG. 8: BALF cytology 1 day after the last challenge with Der f extract in Der f allergic mice immunized twice with rDer p 2.1 or Bet v-1.

### EXAMPLE

### rDer p 2.1 Vaccination in an Allergic Asthma Mouse Model

Material & Methods

Animal Procedures for Allergic Model:

Balb/c female mice 6-8 weeks of age were purchased from Charles River breeding laboratories and used for all experiments. They were housed in UTE IRT-UN platform (Nantes, France). They were maintained under specific pathogen-free, temperature-controlled conditions with a strict 12 hours light-dark cycle and were given free access to food and water. All protocols were approved by the Regional Ethical Committee for Animal Experiments of Pays de la Loire.

Mice were sensitized on days 0, 7, 14 and 21 by percutaneous application of 500 μg of total extract of Dermatophagoïdes pternyssinus (Der p) or Dermatphagoïdes Farinae (Der f) (Stallergenes, Antony, France) in 20 μL of dimethylsulfoxyde (DMSO, Sigma-Aldrich, St. Louis, Mo., United States) on the ears without any synthetic adjuvant. They were challenged intranasally with 250 μg of Der p in 40 μL of sterile PBS, once on D27 to induce asthma and once more on D34 to induce asthma exacerbation.

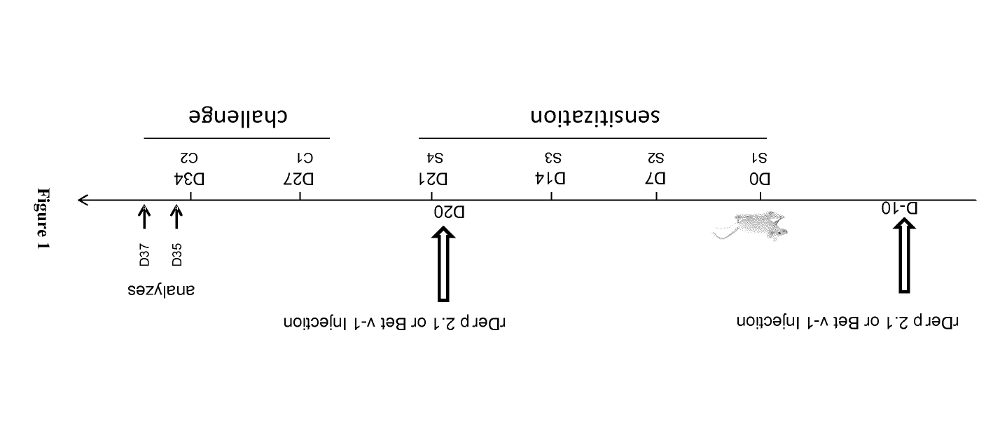

Animals were anesthetized with 100 μl of xylazine (ROMPUN® 2%, Bayer, 15 mg/kg) intraperitoneally and 100 μl of ketamine (IMALGENE®1000, Merial, 80 mg/kg) for both sensitizations and challenges. Mice were sacrificed by sublethal injection of dolethal (DOLETHAL®, vetoquinol, France) on D35 and D37 (FIG. 1). For dynamic lung resistance measurements, mice were anesthetized with 200 μl of a ketamine-xylazine mix and paralyzed with 100 μl of rocuronium bromide (ESMERON®, Organon, 10 mg/ml) intraperitoneally.

Derivative Peptide Injection:

Derivative peptides (Der p 2.1 and Der p 2.2) and control (Bet v 1.2 and Bet v 1.1) peptides were purified as previously described by Chen et al (Mol Immunol, 2008). It should be noted that Bet v 1 is a potent allergen (major white birch (Betula verrucosa) pollen antigen). Recombinant allergen Der p 2 (ref RE-DP2A-1) was obtain from INDOOR biotechnologies.

Peptides were injected 10 days before the first sensitization with Der p and 20 days later. Der p 2.1 and Der p 2.2 were solubilized with 10 mM NaH2PO4, pH 7 solution to a final concentration of 400 μg/ml. Bet v1.1 and Bet v1.2 were used as control and were solubilized with PBS to a final concentration of 150 μg/ml. Native Derp 2 was solubilized with PBS to a final concentration of 1.3 mg/ml. 200 μl of a solution of alu gel (Alu-Gel-S, SERVA Electrophoresis) containing 5 μg of peptides were injected subcutaneously in the neck of mice.

BALF Preparation:

1 mL of sterile PBS was administered intra-tracheally in mice through flexible catheter. Cells and supernatants from removed fluid were separated by centrifugation. Total cell number was determined by optical microscopy and analyze of BALF cell composition was done by Flow cytometry (see below). Supernatants were stored at −20° C. for cytokines assays.

Flow Cytometry Analysis:

Lungs were recovered from asthmatic mice or controls 1 or 3 days after the last challenge and placed into RPMI. Cells were then isolated by passing tissues through 40 μm filter. Cells were first washed with RPMI and next with PBS-FBS 5%. After counting, cells were stained for 20 minutes with titrated specific antibodies in PBS-FBS 5%. The anti-mouse antibodies used included: CD3-APC, CD19 PE-Cy7, CD11c PE-Cy7, F4/80 FITC, CD80 FITC, CD86 PE, Ly6G PerCP-Cy5.5 and CD45R PerCP-Cy5.5 (purchased from eBiosciences); CD8 APC-H7 and CD19 APC-H7 (purchased from BD Biosciences); CCR3 PE (purchased from R&D). Dead cells were excluded using DAPI. Stained cells resuspended in PBS were acquired on a BD LSR™ II (BD Biosciences) and analysed on BD FACSDiva™ software (BD Biosciences). The same protocol was used for BALF cell analysis.

Lung Histology:

1 mL of paraformaldehyde (PFA) 4% was administered intratracheally directly in the lungs through flexible catheter. Trachea was then ligatured and lungs excised. Lungs were fixed in PFA 4% for at least 48 h, embedded in paraffin, cut and stained with periodic acid schiff (PAS) or hematoxylin eosin (H&E) for morphological studies and inflammation scoring. Inflammation scores were calculated from 0 to 7 by adding bronchial epithelial cells dystrophy grade (0 to 3) and peribronchial/perivascular inflammatory infiltrate abundance grade (0 to 4).

Respiratory Function Measurement:

Unrestrained mice were nebulized with increasing doses of methacholine (MCh) (Sigma-Aldrich) from 0 to 40 mg/mL. Enhanced pause (Penh) was measured by whole body plethysmography (Emka Technologies, Paris, France). Dynamic lung resistances were measured using a flexiVent® (SCIREQ). Mice were anesthetized and tracheotomized and placed under mechanical ventilation. Mice were nebulized with increasing doses of methacholine from 0 to 20 mg/mL through a stiff tracheal catheter.

Statistical Analysis:

All statistical analysis were conducted using GraphPad Prism 4.0b (Graphpad Software Inc., La Jolla, Calif. USA). All data are expressed as mean±standard error of the mean (SEM). Unless otherwise specified, differences in airway response between groups were tested for statistical significance using ANOVA. The Mann-Whitney test was used for other analysis. A p value less than 0.05 was considered statistically significant.

Results

Mice Model of Dermatophagoïdes pternyssinus (Der p) Allergic Asthma:

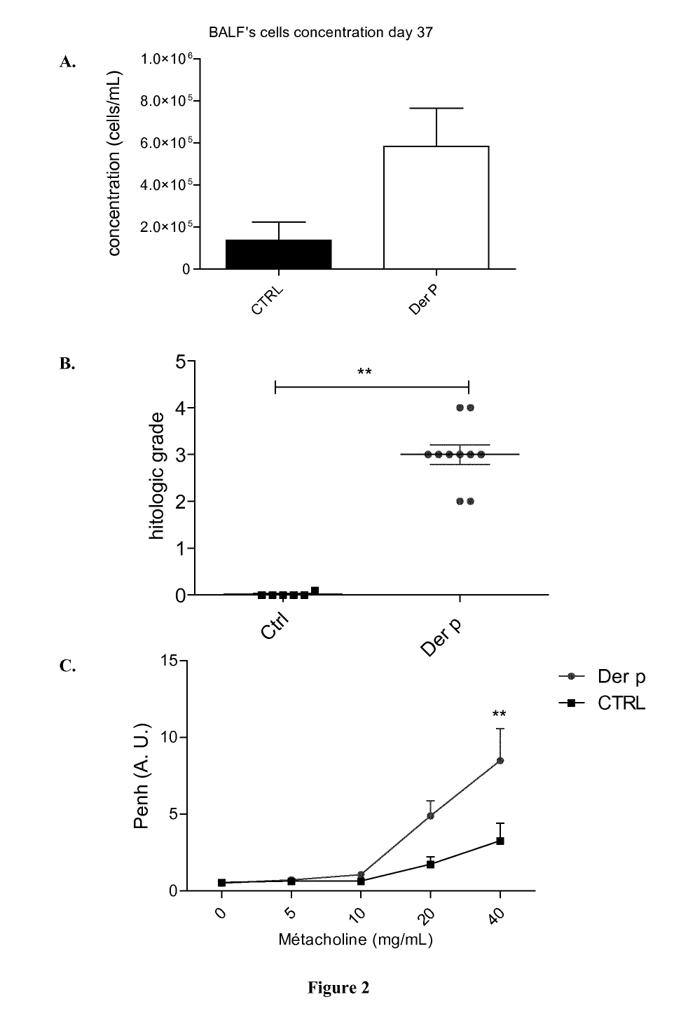

Mice were sensitized on days 0, 7, 14 and 21 by skin application of total extract of DMSO-solubilized Dermatophagoïdes pternyssinus (Der p). In control mice, DMSO alone was used. At D27 and D35, Der p sensitized mice received an intranasal challenge of PBS-solubilized Der p extract or PBS. On Day 37, BALF analysis demonstrated a sustained pulmonary inflammation of Der p sensitized mice compared to controls, with still a three fold increase of BALF cell concentration (FIG. 2A) due to a influx of PMN, Eos and lymphocytes (data not shown). This sustained inflammation was also found at the lung histological levels (FIG. 2B). Indeed histological examination of Der p allergic mice on day 35 revealed an extensive perivascular and peribronchial immune cell infiltration and an epithelial dystrophy with goblet cell hyperplasia (FIG. 2B).

Respiratory function was assessed by Penh measurements in unrestrained mice challenged with increasing doses of nebulized methacholine at 3 day after the second pulmonary challenge with Der p extract or PBS. Experiments were performed in Der p-sensitized and control mice. On day 37, Der p-sensitized and challenged mice displayed a significant increase of respiratory function in response to methacholine compared to the control groups (FIG. 2C).

All together these data show that epicutaneous sensitization followed by two pulmonary challenge with Der p extract induce airway inflammation and modification of respiratory function which mimic allergic asthma.

Immunizations with Recombinant Peptide of Dermatophagoïdes pternyssinus (rDer p 2.1) Improves Respiratory Function:

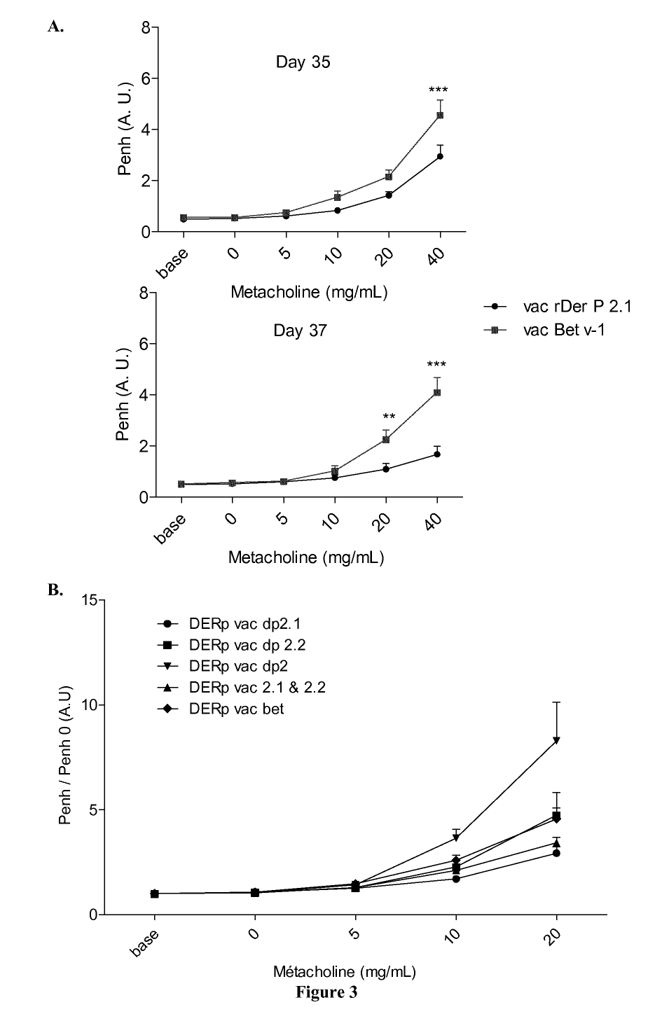

Ten days before starting allergic asthma induction with Der p and 20 days after, 200 μl of a solution containing recombinant peptide of Der p 2 (rDer p 2.1, rDerp 2.2, rDer p 2.1+2.2 or native Der p 2) or control peptide (Bet v-1) was injected subcutaneously with an adjuvant (alu-gel). Analysis 1 and 3 days after the last challenge with Der p extract showed a decrease of Penh in asthmatic mice immunized with rDer p 2.1 (FIG. 3A). This result was observed only in asthmatic mice immunized with rDer p 2.1, asthmatic mice immunized with rDer p 2.2 or rDer p 2.1+2.2 show no significant improvement of respiratory function (FIG. 3B). Surprisingly, immunizations with native Der p 2 shows a degradation of respiratory function compare to control mice.

All together, this data show that immunization with rDer p 2.1 and only this peptide improves the respiratory function in allergic asthmatic mice.

Immunizations with Recombinant Peptide of Dermatophagoïdes pternyssinus (rDer p 2.1) Induce Improvement of Inflammatory Cells Influx in BALF:

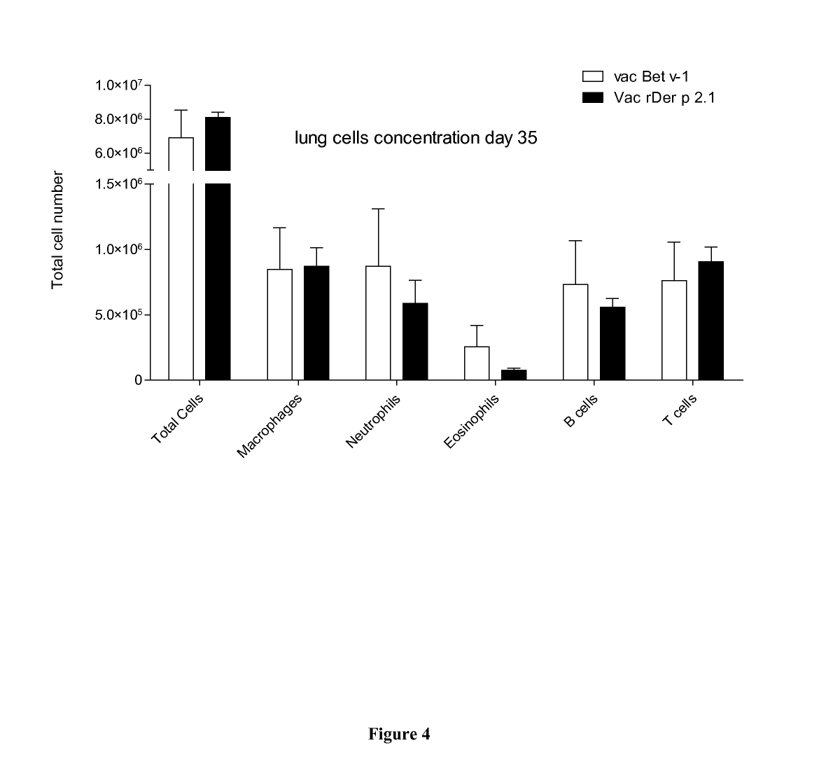

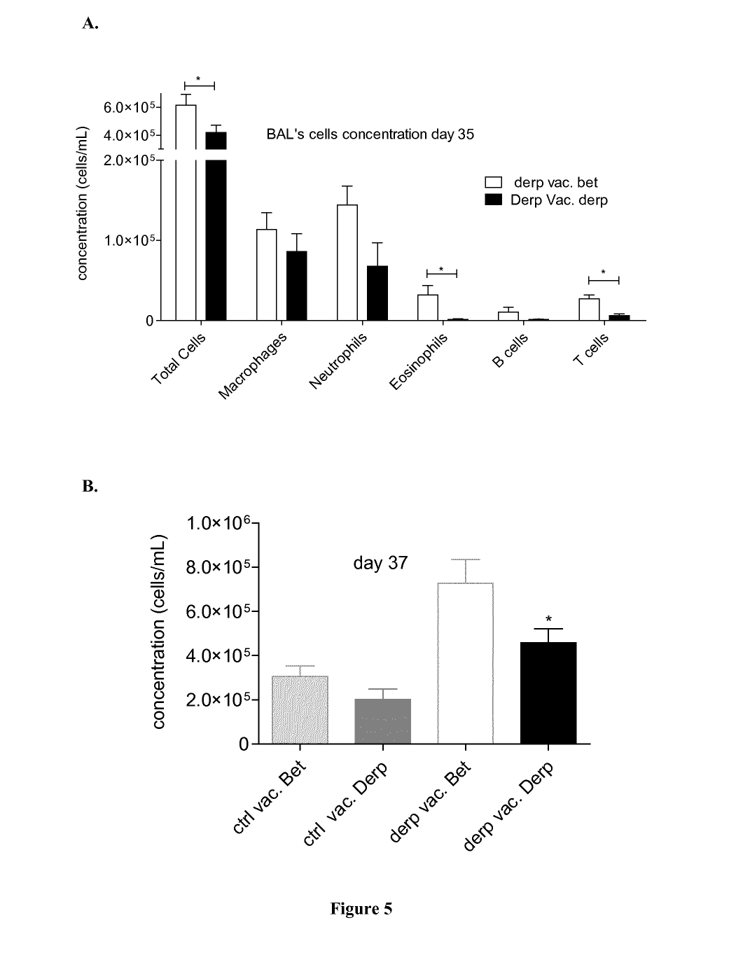

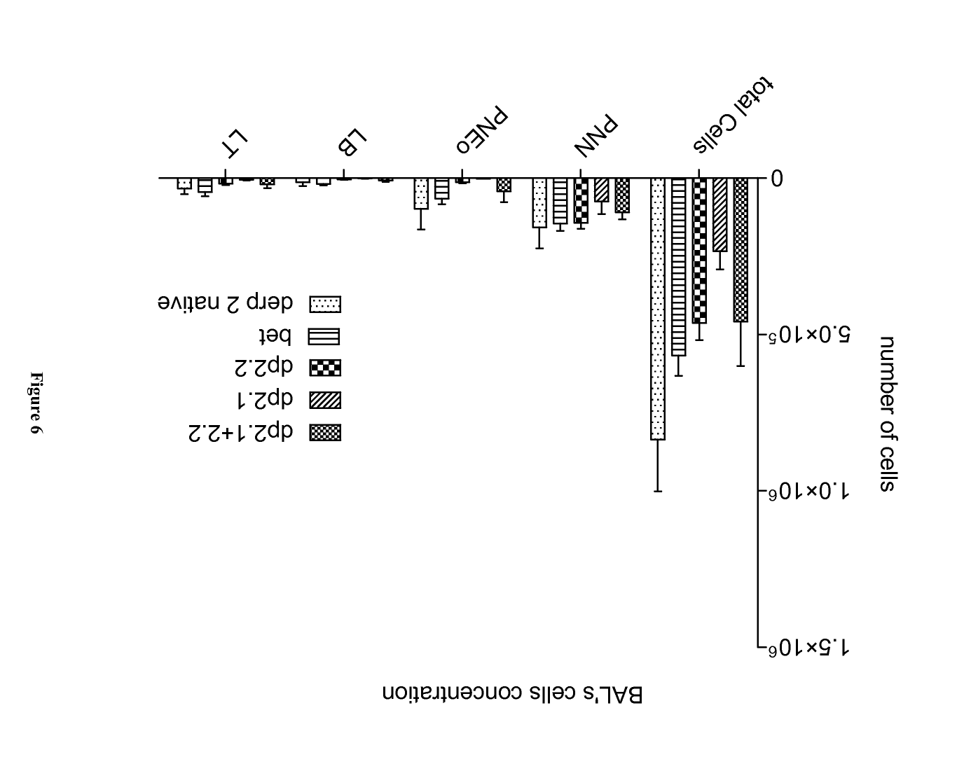

On Day 35, lung cytology was not different between asthmatic mice immunized with rDer p 2.1 or control peptide (FIG. 4), although there was a tendency to a decrease in Eos. In BALF, on day 35 a significant decrease of cell's concentration was observed in asthmatic mice immunized with rDer p 2.1 compare to asthmatic mice immunized with control peptide (FIG. 5A). Immunization with other peptide as rDer p 2.2 or a combination of rDer p2.1+2.2 shows unsignificant tendancy to decrease BALF inflammation (FIG. 6). Vaccinations with native Der p 2 increase BALF inflammation.

On day 37 this significant lower cell's concentration was still observed (FIG. 5B). BALF's cytology was also modulate with a trend to decrease of macrophages, PMN and B cells and a significant decrease of Eos and T cells in allergic asthmatic mice immunized with rDer p 2.1 compared to allergic asthmatic mice immunized with Bet v-1.

All this data indicate that immunization with rDer p 2.1 induce a modification of cells influx on site of lung inflammation with a modulation of cells quantity and cellular composition. All together, this data show that two vaccinations with rDer p 2 improve the respiratory function in allergic mice with a decrease in inflammatory cell influx in BALF.

It should further be noted that immunization with rDer p 2.2 leads to the same results obtained with Bet v-1 (the control peptide) regarding to the different analyzed physiologic parameters (i.e. respiratory function and inflammatory cell influx in BALF).

Immunizations with Recombinant Peptide of Dermatophagoïdes pternyssinus (Der p 2.1) Induce Respiratory Function and Inflammatory Cells Influx in BALF Improvement in Der f Allergic Mice:

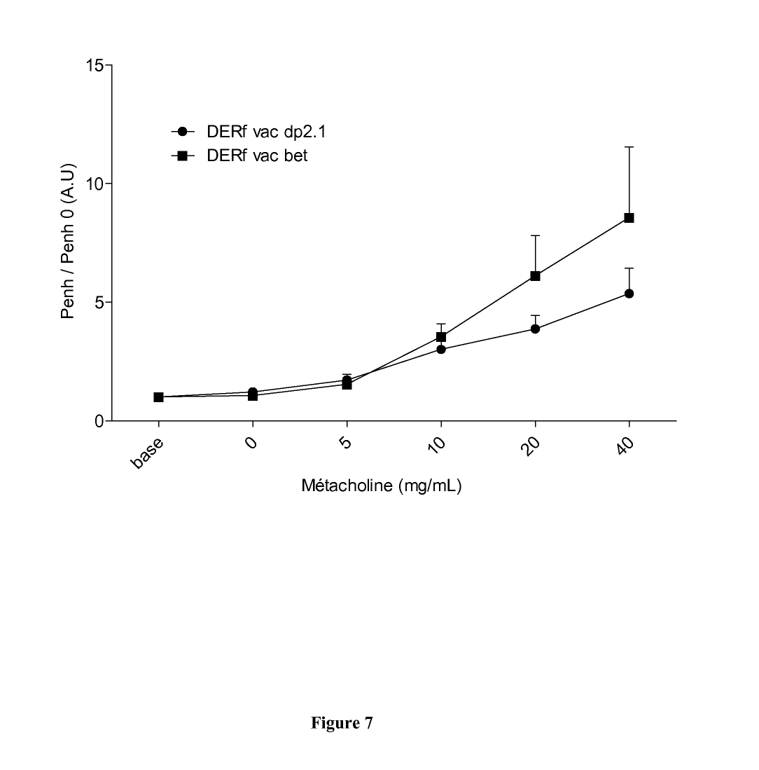

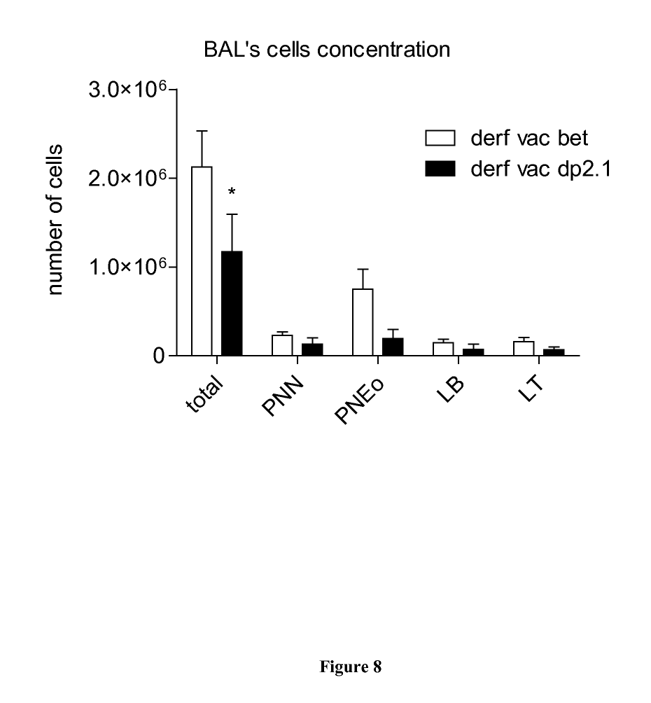

Ten days before starting allergic asthma induction with Der f and 20 days after, 200 μl of a solution containing recombinant peptide of Der p 2 (Der p 2.1) or control peptide (Bet v-1) was injected subcutaneously with an adjuvant (alu-gel). Analysis 1 days after the last challenge with Der f with whole body plethysmography (a non invasive method) showed a trend to modify respiratory function in asthmatic mice immunized with rDer p 2.1 (FIG. 7). This result was correlated with BALF influx. Total cells number in BALF was significantly decreased in mice immunized with rDer p 2.1 compare to mice immunized with Bet v 1. All cells type show a trends to decrease in mice immunized with rDer p 2.1 (FIG. 8). The low number of mice (n=5) and the variability of the protocol could explain that all results are no significant.

All together this data show that immunization with rDer p 2.1 induce airways hyperresponsiveness and inflammatory cells influx in BALF improvement in Der p allergic mice but probably in Der f asthmatic mice too.

### REFERENCES

Throughout this application, various references describe the state of the art to which this invention pertains. The disclosures of these references are hereby incorporated by reference into the present disclosure.
Chen K W, Fuchs G, Sonneck K, Gieras A, Swoboda I, Douladiris N, Linhart B, Jankovic M, Pavkov T, Keller W, Papadopoulos N G, Valent P, Valenta R, Vrtala S; Reduction of the in vivo allergenicity of Der p 2, the major house-dust mite allergen, by genetic engineering; Mol Immunol. 2008 May; 45(9):2486-98.

1. An isolated polypeptide for the prophylactic treatment of allergic asthma in a patient in need thereof comprising a biologically active fragment of at most 100 consecutive amino acids of the allergen of the house-dust mite Dermatophagoides pteronyssinus Der p 2, wherein said fragment comprises:
a) the sequence consisting of amino acids 18 to 70 of SEQ ID NO: 1; or
b) a sequence at least 80% identical to the sequence of (a); or
c) at least six consecutive amino acids of the sequence of (a) or (b).

2. The polypeptide according to claim 1, wherein the polypeptide consists of the sequence consisting of amino acids 18 to 70 of SEQ ID NO: 1.

3. A nucleic acid sequence encoding a polypeptide according to claim 1.

4. A vaccine comprising a polypeptide according to claim 1.

5. (canceled)

6. A vaccine comprising a nucleic acid sequence according to claim 3.

7. A method for the prophylactic treatment of allergic asthma in a patient in need thereof comprising
administering a therapeutically effective amount of an isolated polypeptide, or a nucleic acid encoding said polypeptide, comprising a biologically active fragment of at most 100 consecutive amino acids of the allergen of the house-dust mite Dermatophagoides pteronyssinus Der p 2, wherein said fragment comprises:
a) the sequence consisting of amino acids 18 to 70 of SEQ ID NO: 1; or
b) a sequence at least 80% identical to the sequence of (a); or
c) at least six consecutive amino acids of the sequence of (a) or (b).

8. The method of claim 7 wherein said patient is a mite-allergic patient or an atopic patient without asthmatic manifestation.


SEQUENCE LISTING (1 sequence)

SEQ ID NO: 1 - protein, 146 aa, Artificial Sequence
  Synthetic Der P 2 with a signal peptide
MMYKILCLSLLVAAVARDQVDVKDCANHEIKKVLVPGCHGSEPCIIHRGKPFQLEAVFEANQNTKTAKIEIKASIDGLEVDVPGIDPNACHYMKCPLVKGQQYDIKYTWNVPKIAPKSENVVVTVKVMGDDGVLACAIATHAKIRD